(19) United States
(12) Patent Application Publication
(10) Pub. No.: US 2018/0185251 A1
(43) Pub. Date: Jul. 5, 2018

(54) MASK-PACK KIT

(71) Applicant: THE OOZOO CO., LTD., Seoul (KR)

(72) Inventor: Young Wook CHOI, Seoul (KR)

(21) Appl. No.: 15/567,030

(22) PCT Filed: Apr. 12, 2016
(86) PCT No.: PCT/KR2016/003831

§ 371 Date: Mar. 9, 2018

(30) Foreign Application Priority Data
Apr. 16, 2015 (KR) 10-2015-0053740

(51) Int. Cl. A61K 8/02; A61Q 19/00; B65D 33/00

(52) U.S. Cl. CPC A61K 8/0212; B65D 33/00; A61Q 19/00

(57) ABSTRACT

The present invention relates to a mask-pack kit, and more particularly, to a mask-pack kit capable of uniformly distributing a separately packaged skin improving agent over the whole surface of the skin improving agent when a cosmetic sheet is impregnated in the cosmetic sheet.

### TECHNICAL FIELD

The present invention relates to a mask-pack kit, and more particularly, to a mask-pack kit capable of uniformly distributing a separately packaged skin improving agent over the whole surface of a cosmetic sheet when the skin improving agent is impregnated in the cosmetic sheet.

### BACKGROUND ART

With the development of modern society, men as well as women have paid much attention to skin care. In particular, the interest in preventing a skin from aging while keeping a skin elastic and clear keeps increasing. To keep pace with the trend, various kits for skin improvement have been released in the market.

As the kit for skin improvement, thanks to the convenience, various mask packs have been released in the market. The mask pack is configured to permeate skin improving agents including a nutrient and a moisturizer into a cosmetic sheet. Representatively, there is a mask pack used for a facial skin.

The mask pack is sold while being accommodated in a pouch. For using the mask pack, the user first opens the pouch and then attaches the mask pack to his/her face.

That is, the existing pouch has a cosmetic sheet of a non-woven material and skin improving agents accommodated therein and the cosmetic sheet is moistened with the skin improving agents, and as a result the user opens the pouch and then immediately uses the cosmetic sheet for his/her face.

However, when the user takes out the cosmetic sheet moistened with the skin improving agents by opening the existing pouch, the cosmetic sheet is moistened with the skin improving agents and therefore if the cosmetic sheet is attached to his/her face, the skin improving agents flow out from the cosmetic sheet and thus flow along his/her face, such that the use of the cosmetic sheet may be inconvenient.

Due to the above inconvenience, one example of the mask pack in which the cosmetic sheet and the skin improving agents are separately packaged and then the cosmetic sheet is used by being moistened with a proper amount of skin improving agents is disclosed in Korean Patent No. 10-1176723 (hereinafter, referred to as ‘Related Art Document 1’).

FIG. 1 is a diagram illustrating the existing mask pack according to the related art document 1.

As illustrated in FIG. 1, the mask pack according to the related art document 1 includes a pouch body 10 accommodating a skin improving agent 1, an auxiliary pouch body 20 provided at one side of the pouch body 10, a cosmetic sheet 30 accommodated in the auxiliary pouch body 20 and moistened with the skin improving agent 1 of the pouch body 10, and a scale part 40 having a plurality of scales formed on the pouch body 10.

In this configuration, one side of the pouch body 10 is formed with an outlet 15 through which the skin improving agent 1 is discharged and the outlet 15 is provided with a burst diaphragm 17.

For using the mask pack, the auxiliary pouch 20 is pressurized to burst the burst diaphragm 17, and thus the skin improving agent is introduced into the pouch body to moisten the cosmetic sheet 30.

However, the above-mentioned mask pack has a problem in that an inlet of the skin improving agent is biased to one side and thus the skin improving agent may not be uniformly diffused.

In addition, a central portion of the mask pack requiring a large amount of skin improving agent has reduced distribution, such that there is a problem in that the skin improving agent may not be effectively coated on a face.

### RELATED ART DOCUMENT

### Patent Document

1. Korean Patent No. 10-1176723 (Registered on Aug. 17, 2012)

### DISCLOSURE

### Technical Problem

An object of the present invention is to provide a mask pack capable of uniformly distributing a separately packaged skin improving agent over the whole surface of a cosmetic sheet when the skin improving agent is impregnated in the cosmetic sheet.

Another object of the present invention is to provide a mask pack configured to thickly impregnate a skin improving agent in a sheet corresponding to a facial part at which a concentration of the skin improving agent needs to be high.

### Technical Solution

In one general aspect, a mask-pack kit includes: a pouch configured to have a cosmetic sheet accommodated therein and be provided with an inlet for injecting a skin improving agent into a portion thereof, and an injection means configured to inject the skin improving agent through the inlet of the pouch.

The inlet may be formed on a side of the pouch.

The inlet may be formed within ⅔ of a distance from a center of the side of the pouch to the edge part.

The inlet of the pouch may be inclinedly formed.

The injection means may be a syringe.

The injection means may be a spuit.

The injection means may be an elastic pouch.

The elastic pouch may be connected to the pouch by a string.

The string connecting between the elastic pouch and the pouch may be knotted.

### Advantageous Effects

According to the mask-pack kit in accordance with the exemplary embodiments of the present invention as described above, the inlet may be installed at any place of the pouch, and therefore the skin improving agent may be thickly impregnated in the portion where a large amount of skin improving agent is required.

Further, according to the mask-pack kit in accordance with the exemplary embodiments of the present invention, the side of the pouch may be provided with the inlet to thickly supply the skin improving agent to the portion where the skin improving agent is most likely to be needed.

Further, according to the mask-pack kit in accordance with the exemplary embodiments of the present invention, the inlet is formed within ⅔ of the distance from the center of the side of the pouch to the edge portion, such that the concentration of the skin improving agent around a nose where the skin improving agent is most needed may be improved.

Further, according to the mask-pack kit in accordance with the exemplary embodiments of the present invention, the inlet of the pouch is inclinedly formed, and as a result the skin improving agent introduced through the inlet may be easily diffused to the cosmetic sheet.

Further, the mask-pack kit in accordance with the exemplary embodiments of the present invention may include the syringe as the injection means to forcibly inject the skin improving agent into the pouch.

Further, the mask-pack kit in accordance with the exemplary embodiments of the present invention may include the spuit as the injection means to obtain and use the skin improving agent having the required function from the external container.

Further, the mask-pack kit in accordance with the exemplary embodiments of the present invention may include the elastic pouch as the injection means to pressurize the elastic pouch by hand, thereby easily injecting the skin improving agent into the pouch.

Further, the mask-pack kit in accordance with the exemplary embodiments of the present invention may be variously decorated by connecting the elastic pouch to the pouch with a string.

Further, the mask-pack kit in accordance with the exemplary embodiments of the present invention may be displayed while being hanged to the stand by knotting the string connecting between the elastic pouch and the pouch.

### DESCRIPTION OF DRAWINGS

The above and other objects, features and advantages of the present invention will become apparent from the following description of preferred embodiments given in conjunction with the accompanying drawings, in which:

FIG. 1 is a diagram illustrating the existing mask pack according to the related art document 1;

FIG. 2 is a front view illustrating a mask-pack kit according to a first exemplary embodiment of the present invention;

FIG. 3 is a side view illustrating an inlet of the mask-pack kit according to a first exemplary embodiment of the present invention;

FIG. 4 is a front view illustrating a mask-pack kit according to a second exemplary embodiment of the present invention; and

FIG. 5 is a front view illustrating a mask-pack kit according to a third exemplary embodiment of the present invention.

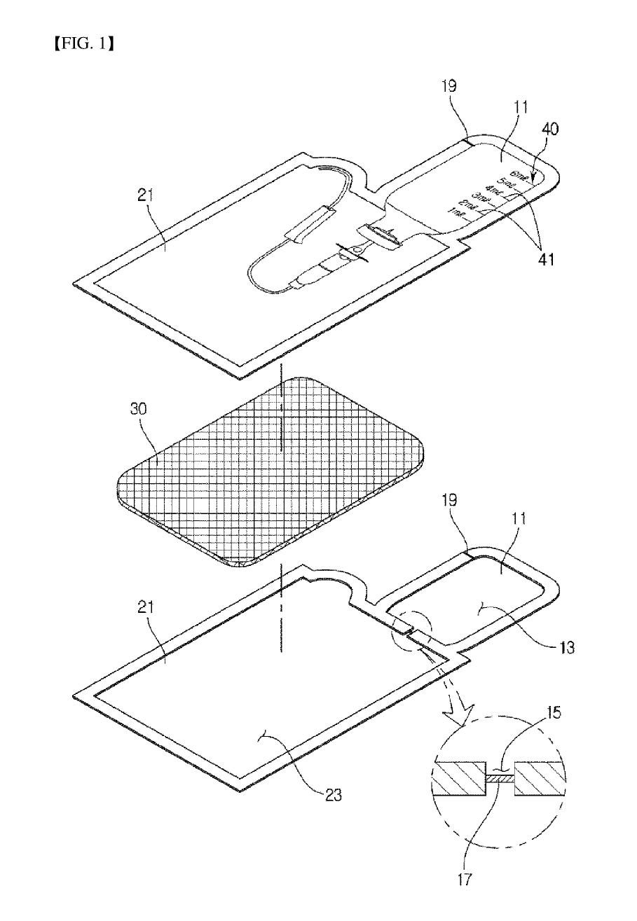

### DETAILED DESCRIPTION OF MAIN ELEMENTS

110, 210, 310: Pouch
120, 220, 320: Injection means
122: Syringe
222: Spuit
322: Elastic pouch

### BEST MODE

Hereinafter, a mask-pack kit according to exemplary embodiments of the present invention will be described below with reference to the accompanying drawings.

### First Exemplary Embodiment

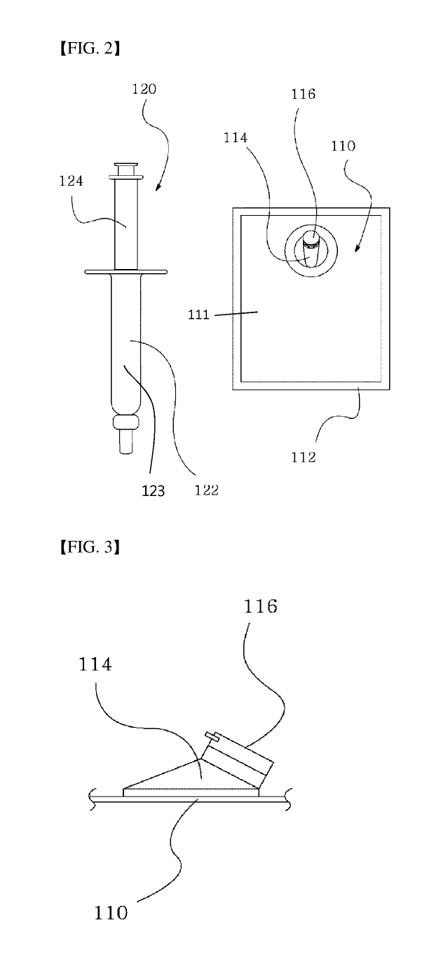

FIG. 2 is a front view illustrating a mask-pack kit according to a first exemplary embodiment of the present invention and FIG. 3 is a side view illustrating an inlet of the mask-pack kit according to a first exemplary embodiment of the present invention.

As illustrated in FIGS. 2 and 3, the mask-pack kit according to the first exemplary embodiment of the present invention includes a pouch 110 having a cosmetic sheet accommodated therein and an injection means 120 for injecting a skin improving agent into the pouch 110.

An inside of the pouch 110 is formed with a space in which the cosmetic sheet (not illustrated) is accommodated. To take out the cosmetic sheet to the outside and make the cosmetic sheet user-friendly, the pouch 110 may be preferably made of a material that can be torn by a user while he/she grips the pouch 110. Further, the pouch 110 may be preferably made of a synthetic resin material so that an edge part 112 thereof may be ultrasonically welded. If necessary, an upper portion of the pouch 110 may be provided with an opening and closing means like a zipper (not illustrated) for reuse to block air from the outside.

A side of the pouch 110 is provided with an inlet 114 for injecting a skin improving agent. The inlet 114 is provided with a lid 116 to be opened and closed. However, if necessary, the pouch 110 may be cut open to be conveniently used as single use only.

The inlet 114 is formed within ⅔ of a distance from a center of the side of the pouch 110 to the edge part 112. Therefore, when the skin improving agent is supplied through the inlet 114, a concentration of the skin improving agent is increased at a nose part that is a central part. Further, the skin improving agent is injected from the center, and therefore may be uniformly diffused to several portions of the cosmetic sheet.

Here, the inlet 114 is inclinedly provided on the side of the pouch 110. In this case, since the inlet 114 is inclinedly formed, a resistance is reduced upon the injection of the skin improving agent, and thus the skin improving agent may be easily injected. Further, the inlet 114 may be preferably formed to be inclined downward toward a central direction. As such, when the inlet 114 is formed to be inclined downward toward the central direction, the skin improving agent is injected toward the center, and thus the skin improving agent may be more uniformly impregnated in several portions of the cosmetic sheet.

Further, according to the first exemplary embodiment of the present invention, the injection means 120 may be a syringe 122. The syringe has the skin improving agent accommodated therein. Therefore, when a piston 124 of the syringe 122 is pressed, the skin improving agent is discharged to the outside. Here, the syringe 122 is used as the injection means 120 to inject the skin improving agent, such that the skin improving agent may be injected at a strong pressure. As a result, the skin improving agent may be easily impregnated up to portions far away from the inlet 114.

### Second Exemplary Embodiment

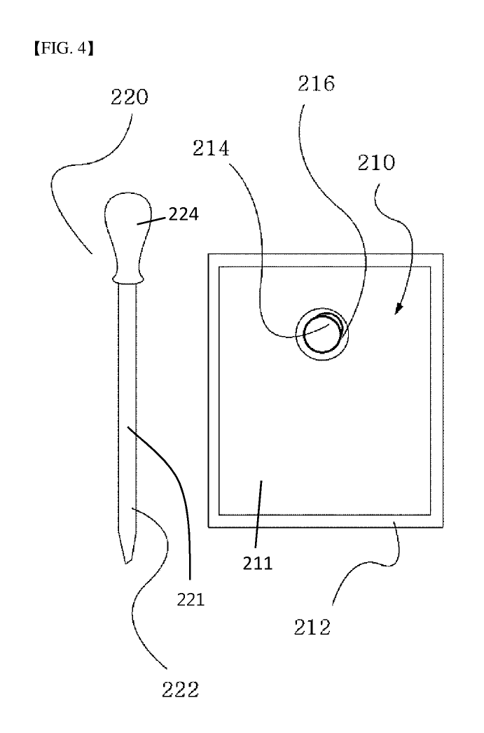

FIG. 4 is a side view illustrating an inlet of the mask-pack kit according to a second exemplary embodiment of the present invention.

As illustrated in FIG. 4, the mask-pack kit according to the second exemplary embodiment of the present invention includes a pouch 210 having a cosmetic sheet accommodated therein and an injection means 220 for injecting a skin improving agent into the pouch 210.

An inside of the pouch 210 is formed with a space in which the cosmetic sheet (not illustrated) is accommodated. To take out the cosmetic sheet to the outside and make the cosmetic sheet user-friendly, the pouch 210 may be preferably made of a material that can be torn by a user while he/she grips the pouch 210. Further, the pouch 210 may be preferably made of a synthetic resin material so that an edge part 212 thereof may be ultrasonically welded. If necessary, an upper portion of the pouch 210 may be provided with an opening and closing means like a zipper (not illustrated) for reuse to block air from the outside.

A side of the pouch 210 is provided with an inlet 214 for injecting a skin improving agent. The inlet 214 is provided with a lid 216 to be opened and closed. However, if necessary, the inlet 214 may be cut open to be conveniently used as single use only.

The inlet 214 is formed within ⅔ of a distance from a center of the side of the pouch 210 to the edge part 212. Therefore, when the skin improving agent is supplied through the inlet 214, a concentration of the skin improving agent is increased at a nose part that is a central part. Further, the skin improving agent is injected from the center, and therefore may be uniformly diffused to several portions of the cosmetic sheet.

Further, according to the second exemplary embodiment of the present invention, the injection means 220 may be a spuit 222. The spuit 222 discharges the skin improving agent filled therein by pressing a pressing part 224 of a rubber material. Here, if necessary, the spuit 222 sucks a skin improving agent having another effect and accommodated in another container (not illustrated) and discharges the skin improving agent and injects the skin improving agent through the inlet 214, thereby using various skin improving agents.

### Third Exemplary Embodiment

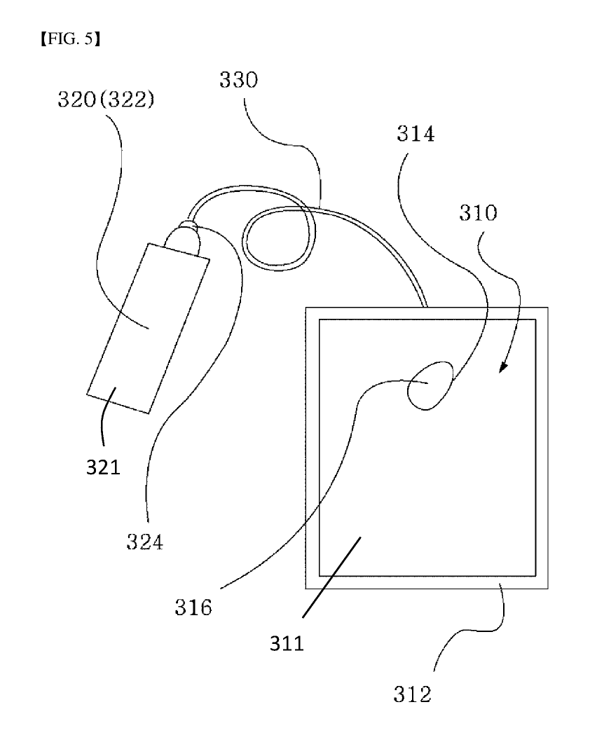

FIG. 5 is a side view illustrating an inlet of the mask-pack kit according to a third exemplary embodiment of the present invention.

As illustrated in FIG. 5, the mask-pack kit according to the third exemplary embodiment of the present invention includes a pouch 310 having a cosmetic sheet accommodated therein and an injection means 320 for injecting a skin improving agent into the pouch 310.

An inside of the pouch 310 is formed with a space in which the cosmetic sheet (not illustrated) is accommodated. To take out the cosmetic sheet to the outside and make the cosmetic sheet user-friendly, the pouch 310 may be preferably made of a material that can be torn by a user while he/she grips the pouch 310. Further, the pouch 310 may be preferably made of a synthetic resin material so that an edge part 312 thereof may be ultrasonically welded. If necessary, an upper portion of the pouch 310 may be provided with an opening and closing means like a zipper (not illustrated) for reuse to block air from the outside.

A side of the pouch 310 is provided with an inlet 314 for injecting a skin improving agent. The inlet 314 is provided with an adhesive sheet 316 to be opened and closed. However, if necessary, the inlet 314 may be cut open to be conveniently used as single use only.

The inlet 314 is formed within ⅔ of a distance from a center of the side of the pouch 310 to the edge part 312. Therefore, when the skin improving agent is supplied through the inlet 314, a concentration of the skin improving agent is increased at a nose part that is a central part. Further, the skin improving agent is injected from the center, and therefore may be uniformly diffused to several portions of the cosmetic sheet.

Further, according to the third exemplary embodiment of the present invention, the injection means 320 may be an elastic pouch 322. The elastic pouch 322 is made of the synthetic resin material like the pouch 310 and therefore the outlet 324 thereof is open by being torn, the opened outlet 324 is positioned at the inlet opened by releasing the adhesive sheet and the side of the elastic pouch 322 is pressed to supply the skin improving agent. When the skin improving agent is injected through the inlet 314, the adhesive sheet 316 is pressed to close the inlet 314, and then the pouch 310 shakes, thereby easily impregnating the skin improving agent in the cosmetic sheet.

The elastic pouch 322 is connected to the pouch 310 by a string 330. The string 330 may be preferably knotted. When the string 330 is knotted, the mask-pack kit may be displayed while being hanged to a stand, and thus the mask-pack kit may be easily displayed.

1. A mask-pack kit, comprising:
a pouch configured to have a cosmetic sheet accommodated therein and is provided with an inlet for injecting a skin improving agent into a portion thereof; and
an injection means configured to inject the skin improving agent through the inlet of the pouch.

2. The mask-pack kit of claim 1, wherein the inlet is formed on a side of the pouch.

3. The mask-pack kit of claim 2, wherein the inlet is formed within ⅔ of a distance from a center of the side of the pouch to an edge part.

4. The mask-pack kit of claim 1, wherein the inlet of the pouch is inclinedly formed.

5. The mask-pack kit of claim 1, wherein the injection means is a syringe.

6. The mask-pack kit of claim 1, wherein the injection means is a spuit.

7. The mask-pack kit of claim 1, wherein the injection means is an elastic pouch.

8. The mask-pack kit of claim 7, wherein the elastic pouch is connected to the pouch by a string.

9. The mask-pack kit of claim 8, wherein the string connecting between the elastic pouch and the pouch is knotted.

10. The mask-pack kit of claim 2, wherein the inlet of the pouch is inclinedly formed.

11. The mask-pack kit of claim 3, wherein the inlet of the pouch is inclinedly formed.

12. The mask-pack kit of claim 2, wherein the injection means is a syringe.

13. The mask-pack kit of claim 3, wherein the injection means is a syringe.

14. The mask-pack kit of claim 2, wherein the injection means is a spuit.

15. The mask-pack kit of claim 3, wherein the injection means is a spuit.

16. The mask-pack kit of claim 2, wherein the injection means is an elastic pouch.

17. The mask-pack kit of claim 3, wherein the injection means is an elastic pouch.
